(19) United States
(12) Patent Application Publication
(10) Pub. No.: US 2025/0161604 A1
(43) Pub. Date: May 22, 2025

(54) LUNG FUNCTION MONITORING SYSTEM

(71) Applicant: THE ROYAL INSTITUTION FOR THE ADVANCEMENT OF LEARNING/MCGILL UNIVERSITY, Montréal (CA)

(72) Inventors: Wissam SHALISH, Kirkland (CA); Guilherme Mendes SANT ANNA, Montréal (CA)

(21) Appl. No.: 18/949,579

(22) Filed: Nov. 15, 2024

Related U.S. Application Data
(60) Provisional application No. 63/599,725, filed on Nov. 16, 2023.

(51) Int. Cl. A61M 16/00

(52) U.S. Cl. CPC A61M 16/0003; A61M 2240/00

(57) ABSTRACT

A monitoring system for a lung function monitoring system operable to monitor lung function of a patient and including a bubble continuous positive airway pressure (CPAP). The monitoring system includes a sound sensor having a sensing range, a bubble generator of the bubble CPAP device being adapted to be located within the sensing range of the sensor, and a controller operatively connected to the sound sensor. The controller is configured to: receive a sound signal from the sound sensor, the sound signal being indicative of sound generated by bubbles within liquid contained in a fluid container of the bubble generator; determine, based on the sound signal, a parameter of the sound generated by the bubbles; and determine a property representative of lung function of the patient based on the parameter of the sound generated.

### CROSS-REFERENCE TO RELATED APPLICATIONS

The present application claims priority on United States provisional patent application No. 63/599,725 filed Nov. 16, 2023, the entire content of which is incorporated by reference herein.

### TECHNICAL FIELD

The present disclosure relates generally to devices and methods for generating and delivering continuous positive airway pressure therapy and, more particularly, to devices and methods used to generate and deliver air to infant patients, such as those in a neonatal intensive care unit (NICU).

### BACKGROUND

Bubble continuous positive airway pressure (CPAP) is widely used in neonatal intensive care units (NICU) across the world and has been monumental in supporting spontaneously breathing infants in the NICU. Unique to bubble CPAP, the water bubbling creates chest vibrations through high frequency pressure oscillations that are transmitted to the infant's airway and lungs. Existing bubble CPAP machines are relatively simple and low-cost devices, consisting of an inspiratory limb with a gas source and humidifier, a nasal interface, and an expiratory limb immersed under water at a fixed pressure which creates the bubbling in the water chamber. While the existing bubble CPAP devices are suitable for their intended purposes, improvements are sought.

### SUMMARY

There is provided a smart monitoring system adapted to be used in neonates receiving bubble continuous positive airway pressure, for monitoring lung function of the patient by harnessing the mechano-acoustic properties of bubbling sounds and vibrations to continuously monitor effective bubbling, lung compliance, airway resistance, pressure delivery, etc., and thereby monitor lung function.

There is accordingly provided a lung function monitoring system for monitoring lung function of a patient, comprising: a bubble continuous positive airway pressure (CPAP) device including: a pressurized gas source of a breathable gas containing oxygen; an inspiratory conduit fluidly connected to the pressurized gas source and operatively connectable to a patient for supplying air to the patient; an expiratory conduit fluidly connectable to the patient for receiving exhaled gases that are exhaled by the patient; a bubble generator including a fluid container connected to the expiratory conduit downstream of the patient, the bubble generator configured for generating bubbles in a liquid within the fluid container using the exhaled gases flowing in the expiratory conduit; and a monitoring system having: a sound sensor having a sensing range, the fluid container of the bubble generator located within the sensing range of the sound sensor; and a controller operatively connected to the sound sensor, the controller having a processing unit and a computer-readable medium having instructions stored thereon and executable by the processing unit to cause the processing unit to: receive a sound signal from the sound sensor that is indicative of sound generated by bubbles within the liquid of the fluid container; determine a parameter of the sound generated by the bubbles based on the sound signal; and determine a property representative of the lung function of the patient using the parameter of the sound, the property including lung compliance and/or airway resistance of the patient.

The lung function monitoring system as defined above and described herein further includes, in certain embodiments, one or more of the following features, in whole or in part, and in any combination.

In certain embodiments, the parameter of the sound generated by the bubbles includes an amplitude and/or a frequency of the sound generated.

In certain embodiments, the parameter of the sound generated by the bubbles includes frequency oscillation of the sound, the frequency oscillation being representative of pressure oscillations produced by the lung function monitoring system and the patient.

In certain embodiments, the computer-readable medium comprises instructions stored thereon executable by the processing unit to: determine one or more dominant frequencies of the bubbles and/or the exhaled gases flowing in the expiratory conduit.

In certain embodiments, the computer-readable medium comprises instructions stored thereon executable by the processing unit to cause the processing unit to: determine the bubbling status based on the parameter, the bubbling status indicative of adequate bubbling when the parameter is within a desired range and indicative of inadequate bubbling when the parameter is outside the desired range.

In certain embodiments, the computer-readable medium comprises instructions stored thereon executable by the processing unit to: issue an alert when the bubbling status is indicative of inadequate bubbling.

In certain embodiments, the sound sensor includes a microphone, an accelerometer configured to capture vibrations, or a micro-electro-mechanical system (MEMS) microphone.

In certain embodiments, the sound sensor is attached directly to the fluid container of the bubble generator.

In certain embodiments, a second sound sensor is oriented towards an environment outside the fluid container of the bubble generator, the computer-readable medium further comprising instructions stored thereon executable by the processing unit to cause the processing unit to: receive the sound signal from the sound sensor and a second sound signal from the second sound sensor, the second sound signal indicative of ambient noises generated in the environment; and correct the sound signal received from the sound sensor using the second sound signal to obtain a corrected sound signal in which the ambient noises are substantially removed from the sound signal.

In certain embodiments, the computer-readable medium comprises instructions stored thereon executable by the processing unit to cause the processing unit to: communicate and continuously update the property representative of the lung function of the patient in real time.

In certain embodiments, the computer-readable medium comprises instructions stored thereon executable by the processing unit to cause the processing unit to: monitor the lung function of the patient over a period of time, by determining a change in the parameter of the sound generated by the bubbles over the period of time, and therefore a change in the property representative of the lung function over the period of time.

There is also provided a monitoring system for a lung function monitoring system, the lung function monitoring system being operable to monitor lung function of a patient and including a bubble continuous positive airway pressure (CPAP), the monitoring system comprising: a sound sensor having a sensing range, a bubble generator of the bubble CPAP device being adapted to be located within the sensing range of the sensor; and a controller operatively connected to the sound sensor, the controller having a processing unit and a computer-readable medium having instructions stored thereon and executable by the processing unit to cause the processing unit to: receive a sound signal from the sound sensor, the sound signal being indicative of sound generated by bubbles within liquid contained in a fluid container of the bubble generator; determine, based on the sound signal, a parameter of the sound generated by the bubbles; determine a property representative of lung function of the patient based on the parameter of the sound generated; and communicate the property representative of the lung function and/or the parameter of the sound generated by the bubbles.

The monitoring system as defined above and described herein further includes, in certain embodiments, one or more of the following features, in whole or in part, and in any combination.

In certain embodiments, the parameter of the sound generated by the bubbles includes an amplitude and/or a frequency of the sound.

In certain embodiments, the parameter of the sound generated by the bubbles includes frequency oscillation of the sound, the frequency oscillation being representative of pressure oscillations produced by the lung function monitoring system and the patient.

In certain embodiments, the computer-readable medium comprises instructions stored thereon executable by the processing unit to: determine one or more dominant frequencies of the bubbles.

In certain embodiments, the sound sensor is a microphone or a micro-electro-mechanical system (MEMS) microphone.

In certain embodiments, a second sound sensor oriented towards an environment outside the fluid container of the bubble generator, the computer-readable medium further comprising instructions stored thereon executable by the processing unit to cause the processing unit to: receive the sound signal from the sound sensor and a second sound signal from the second sound sensor, the second sound signal indicative of ambient noises generated in the environment; and correct the sound signal received from the sound sensor using the second sound signal to obtain a corrected signal in which the ambient noises are substantially removed from the sound signal.

There is further provided a method of monitoring lung function of a patient using a bubble continuous positive airway pressure (CPAP) device, the method comprising: capturing a sound signal representative of sound produced by bubbles within a liquid of a fluid container of the bubble CPAP device, the bubbles being generated in the liquid by exhaled gases from the patient; processing the sound signal to determine a parameter of the sound produced by the bubbles; and determining a property representative of the lung function of the patient using the parameter of the sound generated by the bubbles, the property including lung compliance and/or airway resistance of the patient.

The method as defined above and described herein further includes, in certain embodiments, one or more of the following features and/or steps, in whole or in part, and in any combination.

In certain embodiments, the method includes measuring a frequency oscillation of the sound signal, and determining pressure oscillations present in the exhaled gases of the patient based on the frequency oscillation of the sound signal.

In certain embodiments, the method includes determining: one or more dominant frequencies of the sound signal produced by the bubbles; an amplitude of the sound signal produced by the bubbles; and/or a frequency of the sound signal produced by the bubbles.

In an alternate aspect, there is provided a bubble continuous positive airway pressure (CPAP) device, comprising: a pressurized gas source of a breathable gas containing oxygen; an inspiratory conduit fluidly connected to the pressurized gas source and operatively connectable to a patient for supplying air to the patient; an expiratory conduit fluidly connectable to the patient for receiving gases exhaled by the patient; a bubble generator including a fluid container connected to the expiratory conduit downstream of the patient, the bubble generator configured for generating bubbles in a liquid within the fluid container using the gases flowing in the expiratory conduit; and a monitoring system having: a sensor having a sensing range, the bubble generator located within the sensing range of the sensor; and a controller operatively connected to the sensor, the controller having a processing unit and a computer-readable medium having instructions stored thereon and executable by the processing unit to cause the processing unit to: receive a signal from the sensor, the signal indicative of a phenomenon produced by bubbles generated by the bubble generator; determine a parameter of the phenomenon caused by the bubbles based on the signal; and communicate the parameter and/or a bubbling status determined from the parameter.

The bubble CPAP device as defined above and described herein may also include any one or more of the following features, in whole or in part, and in any combination.

In some embodiments, the computer-readable medium comprises instructions stored thereon executable by the processing unit to cause the processing unit to determine the bubbling status based on the parameter, the bubbling status indicative of adequate bubbling when the parameter is within a desired range and indicative of inadequate bubbling when the parameter is outside the desired range.

In some embodiments, an alert is issued when the bubbling status is indicative of inadequate bubbling.

In some embodiments, the phenomenon is a sound generated by the bubbling, the sensor being a sound sensor, the parameter of the bubbles being an amplitude of a sound generated by the bubbles, the computer-readable medium comprising instructions stored thereon executable by the processing unit to cause the processing unit to issue the alert when the amplitude is below an amplitude threshold.

In some embodiments, the sensor is a microphone or a micro-electro-mechanical system (MEMS) microphone.

In some embodiments, the sound sensor is attached to the fluid container of the bubble generator.

In some embodiments, the phenomenon is vibrations generated by the bubbles on the fluid container, the sensor being an accelerometer configured to capture vibrations generated by the bubbles on the fluid container of the bubble generator.

In some embodiments, a second sensor is oriented towards an environment outside the fluid container of the bubble generator, the computer-readable medium further comprising instructions stored thereon executable by the processing unit to cause the processing unit to: receive a first signal from the sensor and a second signal from the second sensor, the second signal indicative of ambient noises generated in the environment; and correct the first signal received from the sensor using the second signal to obtain the signal in which the ambient noises are substantially removed from the first signal.

In an alternate aspect, there is provided a monitoring system for a bubble continuous positive airway pressure (CPAP) device having a bubble generator including a fluid container, comprising: a sensor having a sensing range, the bubble generator located within the sensing range of the sensor; and a controller operatively connected to the sensor, the controller having a processing unit and a computer-readable medium having instructions stored thereon and executable by the processing unit to cause the processing unit to: receive a signal from the sensor, the signal indicative of a phenomenon produced by bubbles generated within the fluid container of the bubble generator; determine, based on the signal, a parameter of the phenomenon caused by the bubbles; and communicate the parameter and/or a bubbling status determined from the parameter.

The monitoring as defined above and described herein may also include any one or more of the following features, in whole or in part, and in any combination.

In some embodiments, the computer-readable medium comprises instructions stored thereon executable by the processing unit to cause the processing unit to determine the bubbling status based on the parameter, the bubbling status indicative of adequate bubbling when the parameter is within a desired range and indicative of inadequate bubbling when the parameter is outside the desired range.

In some embodiments, an alert is issued when the bubbling status is indicative of inadequate bubbling.

In some embodiments, the phenomenon is a sound generated by the bubbling, the sensor being a sound sensor, the parameter of the bubbles being an amplitude of a sound generated by the bubbles, the computer-readable medium comprising instructions stored thereon executable by the processing unit to cause the processing unit to issue the alert when the amplitude is below an amplitude threshold.

In some embodiments, the sensor is a microphone or a micro-electro-mechanical system (MEMS) microphone.

In some embodiments, the sound sensor is attached to a fluid container of the bubble generator, the fluid container containing a liquid.

In some embodiments, the phenomenon is vibrations generated by the bubbles on a fluid container of the bubble generator, the sensor being an accelerometer configured to capture vibrations generated by the bubbles within the fluid container of the bubble generator.

In some embodiments, a second sensor is oriented towards an environment outside a fluid container of the bubble generator, the computer-readable medium further comprising instructions stored thereon executable by the processing unit to cause the processing unit to: receive a first signal from the sensor and a second signal from the second sensor, the second signal indicative of ambient noises generated in the environment; and correct the first signal received from the sensor using the second signal to obtain the signal in which the ambient noises are substantially removed from the first signal.

In an alternate aspect, there is provided a method for monitoring a bubble continuous positive airway pressure (CPAP) device having a bubble generator, the method comprising: receiving a signal from a sensor, the signal indicative of a phenomenon produced by bubbles generated by the bubble generator; determining, based on the signal, a parameter of the phenomenon caused by the bubbles; and communicating the parameter and/or a bubbling status determined from the parameter.

The method as defined above and described herein may also include any one or more of the following features and/or steps, in whole or in part, and in any combination.

In some embodiments, the method includes determining the bubbling status based on the parameter, the bubbling status indicative of adequate bubbling when the parameter is within a desired range and indicative of inadequate bubbling when the parameter is outside the desired range.

In some embodiments, the method includes issuing an alert when the bubbling status is indicative of inadequate bubbling.

In some embodiments, the phenomenon is a sound generated by the bubbling, the sensor being a sound sensor, the parameter of the bubbles being an amplitude of a sound generated by the bubbles, the method comprising issuing the alert when the amplitude is below an amplitude threshold.

In some embodiments, the sensor is a microphone or a micro-electro-mechanical system (MEMS) microphone.

In some embodiments, the sound sensor is attached to a fluid container of the bubble generator, the container containing a liquid.

In some embodiments, the sensor is an accelerometer configured to capture vibrations generated by the bubbles in a fluid container of the bubble generator.

In some embodiments, the method includes receiving a signal from a second sensor oriented towards an environment outside a fluid container of the bubble generator, the method comprising: receive a first signal from the sensor and a second signal from the second sensor, the second signal indicative of ambient noises generated in the environment; and correct the first signal received from the sensor using the second signal to obtain the signal in which the ambient noises are substantially removed from the first signal.

### DESCRIPTION OF THE DRAWINGS

Reference is now made to the accompanying figures in which:

FIG. 1 is a schematic view of a lung function monitoring system including a bubble continuous positive airway pressure (CPAP) device;

FIG. 2 is a schematic view of a monitoring system of the lung function monitoring system of FIG. 1;

FIG. 3 is an exemplary display for the monitoring system of FIG. 2;

FIG. 4A is a graph illustrating a correlation between an amplitude of sounds generated by bubbles of the device of FIG. 1 and pressures of air supplied to a patient while using the device of FIG. 1;

FIG. 4B is a graph illustrating a correlation between an amplitude of sounds generated by the bubbles of the device of FIG. 1 and oscillations in the pressure of the air supplied to the patient;

FIG. 4C is a graph illustrating a correlation between predicted mean pressures of the air supplied to the patient and measured mean pressures of the air supplied to the patient;

FIG. 4D is a graph illustrating a correlation between predicted pressure oscillations of the air supplied to the patient and measured pressure oscillations of the air supplied to the patient;

FIG. 5 is a flowchart illustrating a step of a method of monitoring the device of FIG. 1;

FIG. 6 is a schematic representation of an exemplary controller for the monitoring system of FIG. 2 for the lung function monitoring system of FIG. 1; and

FIG. 7 is a flowchart illustrating a method of monitoring lung function of a patient using the lung function monitoring system of FIG. 1.

### DETAILED DESCRIPTION

### Lung Function Monitoring System with Bubble CPAP Device

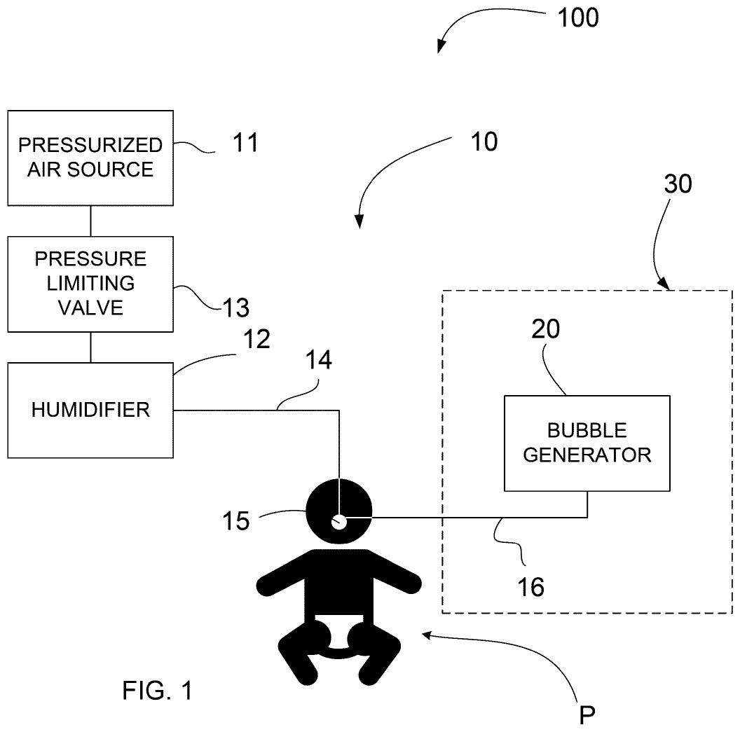

Referring to FIG. 1, a lung function monitoring system 100 is depicted that includes generally a bubble continuous positive airway pressure (CPAP) device 10 and a monitoring system 30. The bubble CPAP device 10 may also be simply referred to in this disclosure as “device” 10. The device 10 is used to provide continuous positive airway pressure therapy to infants. The bubbles generated by the device 10 induce chest vibrations through high frequency pressure oscillations that are transmitted to the infant's airway and lungs. This may have positive effects on development of the patient's lungs. For instance, the bubbling effects on the chest may create high frequency pressure oscillations that may stabilize functional residual capacity, promote alveolar recruitment, and decrease airway inflammation.

As shown in FIG. 1, the device 10 delivers breathable gas (e.g., oxygen, air, etc.) under pressure from a pressurized gas source 11 and via a humidifier 12 used to increase a relative humidity of the air provided to a patient P, herein an infant. The pressurized gas source 11 may include a gas outlet located in a hospital room. A compressor may feed compressed gas to this outlet. The outlet may be configured to supply the gas at a plurality of flow rates (e.g., 6, 8, 10 L/min). The pressurized gas source 11 may be a reservoir of oxygen fluidly connected to a blender to mix the oxygen with air prior to feeding this mixture to the patient. Any suitable source of pressurized gas may be used. In some cases, 100% oxygen or air at any suitable oxygen concentration may be fed to the patient. The humidifier 12 may be omitted in some configurations. The pressurized gas source 11 may be, for instance, a pump or any device able to generate a flow of air at a desired pressure. The pump may be a gear pump, a piston pump, and so on. A flowmeter may be used to control a mass flow rate of air supplied to the patient P. A pressure limiting valve 13 is fluidly connected between the gas source 11 and the patient P, herein between the gas source 11 and the humidifier 12, but other locations are contemplated, for ensuring that a pressure of the air provided to the patient P does not exceed a set threshold. Depending on the pressurized gas source used, the pressure limiting valve 13 and/or the humidifier 12 may be omitted in some embodiments. The air is fed to the patient P via an inspiratory conduit 14 that fluidly connects the pressurized gas source 11 to a nasal interface 15 engaged to the patient P. The inspiratory conduit 14 is also referred to as an inspiratory limb. The nasal interface 15 may be any suitable device able to create a substantially airtight connection with the patient P to maximize air supplied to the patient and to minimize leak. The nasal interface 15 may be a device such as a nasal mask in which small prongs are inserted into the patient's nostrils. Any suitable device known in the art may be used. Gases expelled by the patient P, typically a mixture of air and carbon dioxide, flows through an expiratory conduit 16 via the nasal interface 15. The expiratory conduit 16 is also referred to as an expiratory limb. The air is then fed through a bubble positive airway pressure (PAP) valve, referred to below as a bubble generator 20 located downstream of the patient P and downstream of the nasal interface 15. The bubble generator 20 is configured to generate the bubbles that create the vibrations described above. The lung function monitoring system 100 also includes the monitoring system 30, as will be described in further detail below.

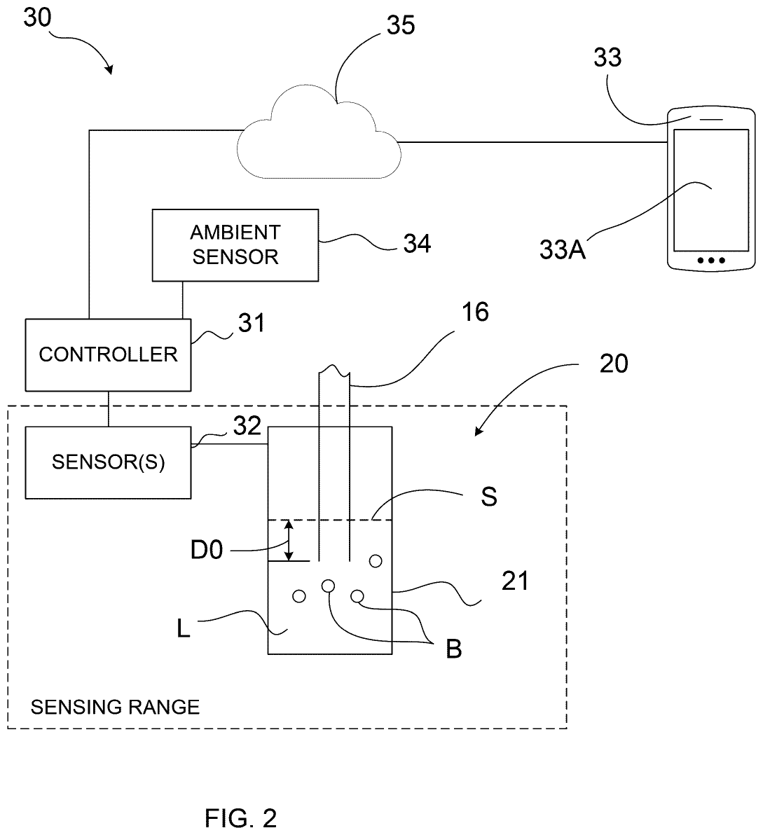

Referring now to FIG. 2, the bubble generator 20 of the bubble CPAP device 10 and the monitoring system 30 will described in more detail. The bubble generator 20 includes a fluid container 21, referred to below simply as a container 21, that contains a liquid L, for example water. A remote end 17 of the expiratory conduit 16 is submerged in the liquid L at a depth D0 below a surface S of the liquid L in the container 21. The depth D0 is selected to adjust the pressure. The greater the depth D0, the greater is the pressure that must be generated by the patient P while exhaling. The pressure may therefore be fixed at a certain value by selecting the depth D0. The depth D0 may be varied by the device 10 to cater to different patients. The bubble generator 20 is configured for generating bubbles in the liquid L within the container 21 using the gases flowing in the expiratory conduit 16.

The device 10 should be substantially leak free for bubbles B to be generated in the liquid L within the container 21. The main source of air leaks is typically at the nasal interface 15 with the patient P. Air may leak out of the device if, for instance, the prongs of the nasal interface 15 are not well adjusted or if they are shifted out of place by movements of the patient P. Leaks may also occur if the patient P opens his or her mouth. In such a case, all of the air will escape through the mouth while bypassing the expiratory conduit 16. In turn, this will cause the bubbles to not be generated since the exhaled air will not reach the bubble generator 20.

Consequently, care must be taken to ensure that the device 10 operates as prescribed. Typically, monitoring of effective bubbling and effective pressure delivery during bubble CPAP is done subjectively by a medical team at the bedside, for example by visual inspection of the water within the container or by auscultating the lungs. This thus involves a hospital employee (e.g., nurse) inspecting the device to determine if bubbles B are adequately generated. If no bubbles are generated, this may indicate that there is a leak between the gas source 11 and the bubble generator 20. This inspection process is time-consuming and care intensive. Moreover, medical personnel cannot assess effective bubbling continuously, and their assessment cannot quantify the degree to which the bubble CPAP device is functioning adequately. Directed continuous monitoring of pressure would require expensive and fragile catheters that require frequent calibration and maintenance. This is thus not a viable solution. Hence, improvements are sought.

### Monitoring System of the Lung Function Monitoring System

The lung function monitoring system 100 is therefore provided with a monitoring system 30, which may at least partially alleviate the aforementioned drawbacks. The monitoring system 30 uses mechano-acoustic properties of sounds and vibrations to estimate effective bubbling and pressure delivery in neonates receiving bubble continuous positive airway pressure (bubble CPAP). It has been found by the inventors of the present disclosure that properties of the bubbles, and more particularly the sounds generated by the bubbles, can be used to monitor effective bubbling and pressure delivery on this mode of respiratory support. More specifically, given that the notion of “bubbling well” is commonly used as a qualitative marker of effective bubble CPAP delivery, it is proposed that continuous recording and analysis of bubbling sounds/vibrations be used to monitor the bubble CPAP device 10.

As shown in FIG. 2, the monitoring system 30 includes a controller 31 operatively connected to a sensor 32. The sensor 32 may include more than one sensor as will be discussed below. The sensor 32 is a sensor able to generate a signal indicative of a phenomenon produced by bubbles generated inside the bubble generator 20 (which may also be referred to as a bubble positive airway pressure valve, or bubble CPAP generator). The phenomenon may include, for instance, noise generated by the bubbles, vibrations generated by the bubbles, fluctuations on a surface S of the liquid in the container 21, fluctuations of a height of the level of the liquid in the container 21, movements on the surface of the liquid, and so on. A parameter of the phenomenon may then be determined based on the signal. The parameter may include, for instance, one or more of an amplitude of the sound generated by the bubbles B, a frequency of the sound generated by the bubbles B, an amplitude or pitch of the vibrations generated by the bubbles B on the container 21, a frequency of the vibrations generated by the bubbles B, a measure of the variations in a shape of the surface S of the liquid (e.g., waves when the bubbles B reach the surface S), a measure of the fluctuation in the height of the surface S caused by the bubbles B reaching the surface S, a measure of the movements on the surface S of the liquid, a wavelength of the sound generated by the bubbles B, and so on.

In one particular embodiment, the sensor 32 includes, for instance, a contact microphone or micro-electro-mechanical system (MEMS) microphone. In another embodiment, the sensor 32 may also include an accelerometer, for example a tri-axial accelerometer embedded within an acoustic sensor, which captures vibrations generated by the bubbles B as well as the sound generated by the bubbles B. The accelerometer is configured to capture vibrations generated by the bubbles on the container 21 of the valve. The accelerometer may be located proximate the surface S of the liquid. The sensor 32 may be a motion sensor able to detect movements on the surface S of the liquid caused by the bubbles B. In some embodiments, the ambient sensor 34 operatively connected to the controller 31 may be used to factor out ambient noise. For instance, the sensor 32 may be oriented towards the liquid in the container 21 to captures noise/vibrations generated by the bubbles B and the ambient sensor 34 may be oriented towards an environment outside the container 21 to capture ambient noises such as people talking, noises generated by other machines, ventilation noises, etc. The signal of both sensors may be combined using known techniques to factor out ambient noise. Any combinations and any numbers of the above sensor 32 may be used.

The sensor 32 may have a sensing range, which may be defined as a distance or range over which the sensor 32 is able to effectively detect, measure, or respond to a specific parameter or stimulus. The specific sensing range depends on the type of sensor and its intended application. Different sensors are designed to sense various parameters, such as temperature, pressure, proximity, motion, light, sound, and more, and their sensing ranges can vary. The sensor 32 used is placed proximate to the bubble generator 20 such that the sensor 32 is able to sense one or more of sounds and vibrations created by the bubbles B. Put differently, the bubble generator 20, or more particularly at least the container 21 of the bubble generator 20, is located within the sensing range of the sensor 32. The sensing range is shown with a dashed line in FIG. 2. The sensor 32 may be affixed to a wall of the container 21, for instance. The sensor 32 may also be located inside or outside the container 21.

The sensor 32 may have a sensitivity selected to capture the parameter of the bubbles B. For instance, when the sensor 32 is a sound sensor, such as a microphone, the sensitivity may be regarded as a measure of how well the sensor 32 converts sound pressure (acoustic energy) into an electrical signal. It is usually expressed in decibels per Pascal (dB/Pa). A higher sensitivity indicates that the microphone can capture quieter sounds, while a lower sensitivity microphone may require louder sounds for the same level of signal output. Sensitivity is a measure of the microphone's range in terms of sound pressure level (SPL) it can pick up. The sensitivity of the microphone is selected so that the microphone is able to detect the sounds generated by the bubbles B within its sensing range.

In a particular embodiment, the sensor 32 is a sound sensor, i.e. an acoustic sensor, also known as an acoustic transducer, which is designed to detect and convert sound or acoustic waves into an electrical signal. These sensors are used in various applications to capture, measure, or monitor sound or vibration in the surrounding environment. Microphones are a subset of acoustic sensors. Microphones come in various types, such as condenser microphones, dynamic microphones, and electret microphones. The sensor 32 may be a piezoelectric sensor. A piezoelectric sensor is able to use the piezoelectric effect to convert mechanical vibrations or acoustic waves into electrical signals. The sensor 32 may be an ultrasonic sensor configured to emit and receive high-frequency sound waves (ultrasonic waves) to measure distances and detect objects. They are widely used in industrial automation and robotics for proximity sensing. The sensor 32 may be a hydrophone. Hydrophones are specialized acoustic sensors designed to detect underwater sounds. Such a hydrophone may be disposed within the liquid in the container 21.

The controller 31 may be operatively connected to a displaying device 33, which may be a smartphone, a tablet, a computer, a screen, and so on. In the embodiment shown, the connection between the controller 31 and the displaying device 33 may be wireless and may be affected through a cloud 35. Alternatively, a direct connection between the controller 31 and the displaying device 33 may be used. Such a direction connection may be made through Bluetooth™, Wi-Fi™, radio transmission, to name a few. Any suitable communication protocol may be used to transmit data between the controller 31 and the displaying device 33. A wired connection may be provided and the displaying device 33 may be located proximate the patient P. The controller 31 may be configured to emit an alarm (e.g., sound) when the bubbling deviates from nominal. This alarm may be issued via the displaying device in some embodiments. The alarm may be a visual indicator, a sound notification, and so on.

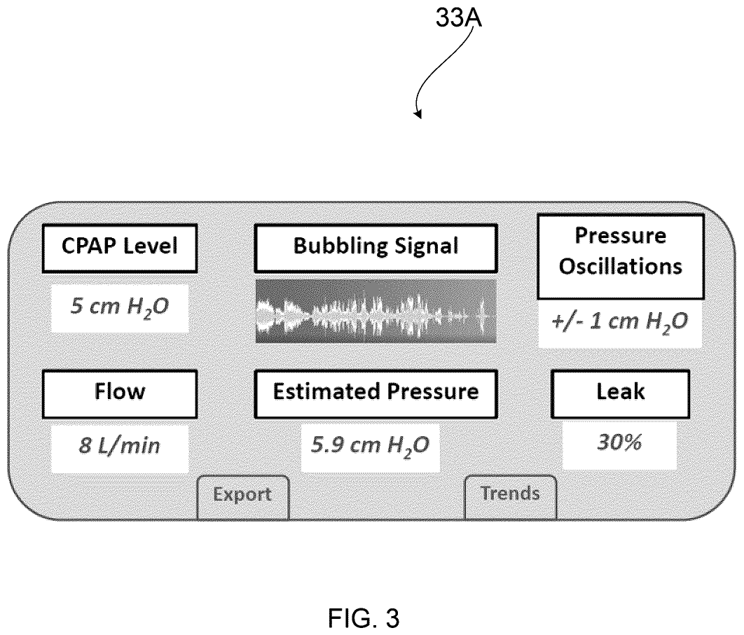

Referring now to FIG. 3, a possible embodiment of a display 33A of the displaying device 33 is shown. The display 33A may indicate, for instance, the CPAP level in term of pressure, a graphical representation of the bubbling signal, a value for pressure oscillations, a flow rate, an estimated pressure provided to the patients P, and an estimation of a percentage of the air supplied from the gas source that does not reach the patient P and, thus, that leaked out of the device 10. Other possible representations are possible without departing from the scope of the present disclosure. The displaying device 33 may display one or more of the following parameters: delivered pressures, pressure oscillations, percentage leaks between the CPAP interface and the patient P, and dominant frequencies of the bubbles generated or of exhaled gases flowing in the expiratory conduit on exhalation, which may be used as a surrogate for lung compliance.

The lung function monitoring system 100 accordingly permits function of the patient to be monitored. This is achieved using the monitoring system 30, which receives a sound signal produced by the sound sensor 32 and indicative of the sound generated by the bubbles within the bubble generator 20 of the bubble CPAP device 10. The monitoring system 30 is configured to determiner a parameter of the sound (e.g., an amplitude and/or a frequency of the sounds, and/or a frequency oscillation) based on this sound signal. The monitoring system 30 is thus able to determine a property that is representative of the lung function of the patient using the measured/determined parameter of the sound. The termination of this property may comprise an estimation or prediction of the property, based on the parameter or parameters of the sound signal, which is processed by the system to determine or predict the lung function property. This property of representative of lung function may include, for example, lung compliance and/or airway resistance of the patient. Lung compliance is understood to be the change in volume in the lungs for a given change in transpulmonary or transmural pressure. Airway resistance is understood to be the change in transpulmonary pressure needed to produce a unit flow of gas through the airways of the lung.

Stated differently, the monitoring system 30 of the present lung function monitoring system 100 is configured to capture and process audible sounds produced by the bubble generator of the bubble CPAP device 10 to estimate airway pressure and/or the magnitude of airway pressure oscillations. This constitutes more than simply a binary determination of whether or not bubbling within the liquid of the bubble CPAP device is occurring. Rather, the monitoring system 30 is capable of capturing, measuring and processing audible sound generated by the bubbles of the bubble CPAP device 10, and determine (albeit indirectly) lung function of the patient by determining, or at least estimating, lung compliance and/or airway resistance of the patient that is generating the bubbles. Accordingly, the acoustic properties of the sounds generated by the bubbles of the bubble CPAP device 10 can act as surrogate instruments to estimate, and monitor, the lung function of the patient and therefore other characteristics such as lung recruitment status. The lung function monitoring system 100 can be thus used after birth to characterize the degree of lung reabsorption in babies receiving bubble CPAP for transient tachypnea of the newborn (TTN). The lung function monitoring system 100 can also be used to indicate the severity of certain lung diseases after birth, including respiratory distress syndrome (RDS) and bronchopulmonary dysplasia. The lung function monitoring system 100 can also be used to measure and determine improvement of such lung conditions in a patient.

As noted above, the monitoring system 30 of the lung function monitoring system 100 is capable of determining a parameter of the sound generated by the bubbles. This parameter can include, in certain embodiments, an amplitude and/or a frequency of the sound generated. Additionally or alternately, the parameter of the sound generated by the bubbles may include a frequency oscillation of the sound, the frequency oscillation being representative of pressure oscillations produced by the lung function monitoring system and the patient. The monitoring system 30 is also capable of determining one or more dominant frequencies of the bubbles and/or the exhaled gases flowing in the expiratory conduit of the bubble CPAP device 10.

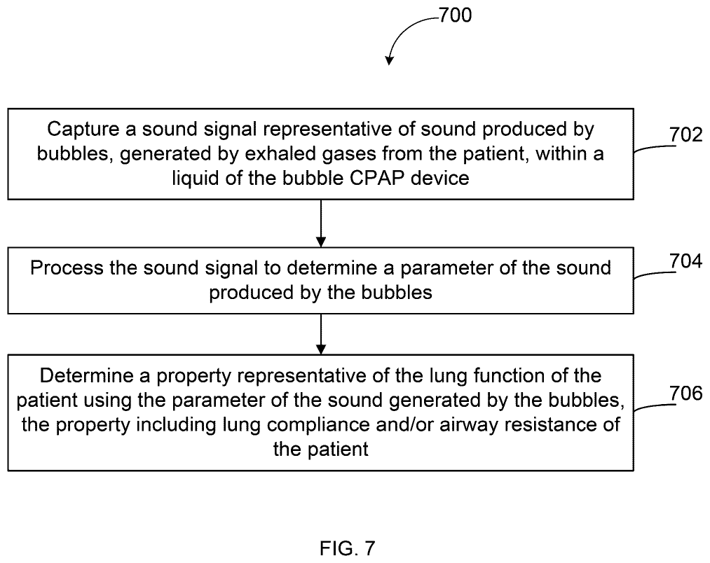

Referring now to FIG. 7, the lung function monitoring system 100 accordingly may be used as part of a method 700 of monitoring lung function of a patient using a bubble CPAP device. This method 700 includes, at 702, capturing a sound signal representative of sound produced by bubbles within a liquid of a fluid container of the bubble CPAP device, the bubbles being generated in the liquid by exhaled gases from the patient. At 704, the method further includes processing the sound signal to determine a parameter of the sound produced by the bubbles. At 706, the method includes determining a property representative of the lung function of the patient using the parameter of the sound generated by the bubbles, the property including lung compliance and/or airway resistance of the patient.

The method 700 and the associated lung function monitoring system 100 permit parameters such as frequency and/or amplitude of a sound generated by the bubbles within the bubble CPAP device 10 to be used to determine a property, such as lung compliance and/or airway resistance of the patient, that is representative of the lung function of the patient. The lung function of the patient can thus be evaluated and monitored, without requiring any invasive devices and without the need to directly measure pressure within the bubble CPAP device. Given that pressure oscillations transmitted to the lungs during bubble CPAP are typically influenced by both the properties of the CPAP system itself (pressure, flow, leaks etc.) and the mechanical characteristics of the lung (e.g. lung compliance), the present system and method uses measurements of sounds generated by the bubbles of the bubble CPAP, instead of measurements of pressure directly measured within the CPAP system, to estimate and ultimately determine changes in lung compliance (by monitoring for example the magnitude of the power of the sound signal at different frequency ranges). At lower lung compliances, the majority of the power in the sound signal may be contained, whereas at higher sound frequencies, and when lung compliance improves, the power of the sound signal may shift back to lower frequencies. When lung compliance is lower, higher frequencies of the sound signal may be more dominant, but with a wider range of pressure fluctuations and higher measured peak pressures. The system is thus configured to monitor the sound signals and watch for such indications of lower lung compliance (and, in certain embodiments, communicate this information, and/or alert a user when appropriate). The present system and method is also configured to determine increased airway resistance in the patient (which can be seen in conditions such as bronchopulmonary dysplasia) based on specific acoustic signatures detected based on the amplitude and frequency characteristics of the bubbling sounds.

In certain embodiments, the parameters and/or the bubbling status may be monitored and displayed in real-time. Herein, the expression “real-time” refers to a mode of computer operation in which data processing, communication, or control functions are performed instantly as they occur, without any noticeable delay. In a real-time system, responses to inputs or events are provided within a predetermined and predictable timeframe, typically within milliseconds or microseconds, ensuring that the system can respond to and interact with the environment or users with minimal or imperceptible delay. Accordingly, the property representative of the lung function of the patient that is determined by the monitoring system 30 may be communicated and continuously updated in real team. In certain embodiments, the monitoring system 30 configured to monitor the lung function of the patient over a period of time, by determining a change in the parameter of the sound generated by the bubbles over the period of time, and therefore a change in the property representative of the lung function over the period of time.

The disclosed monitoring system may thus provide a smarter monitoring system, capable of continuously displaying in real time the delivered pressures, pressure oscillations, and leaks in the CPAP device 10. The potential implications of this might alert respiratory therapists to leaks, or partial obstruction in the circuit to optimize the therapy we are giving.

### Data

To evaluate the relationship between sound properties and pressure, the delivered pressures have been monitored using a pressure transducer inserted at the inspiratory conduit 14 of the bubble CPAP device 10. The following metrics from the bubble sounds and vibrations have been evaluated: root mean square of the audio signal, total power of the audio signal contained between 0 and 7,500 Hz, variance of the audio signal, variance of the frequencies contained in the audio signal, and dominant frequencies contained in the audio signal.

In a controlled experimental environment on a manikin, a very strong correlation between measures of sound or vibration amplitude (RMS, total power) and delivered pressures and pressure oscillations has been found by the inventors of the present disclosure. By demonstrating an excellent correlation between the properties of bubbling sounds/vibrations and delivered pressures and pressure oscillations, the solution proposed in this disclosure may provide continuous monitoring of infants receiving bubble CPAP.

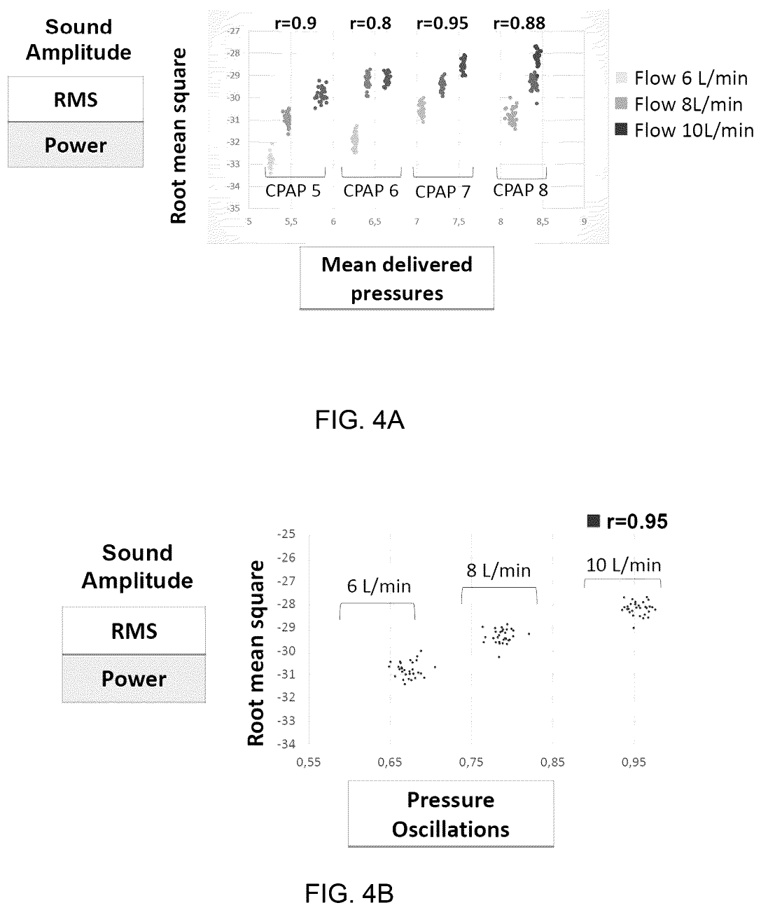

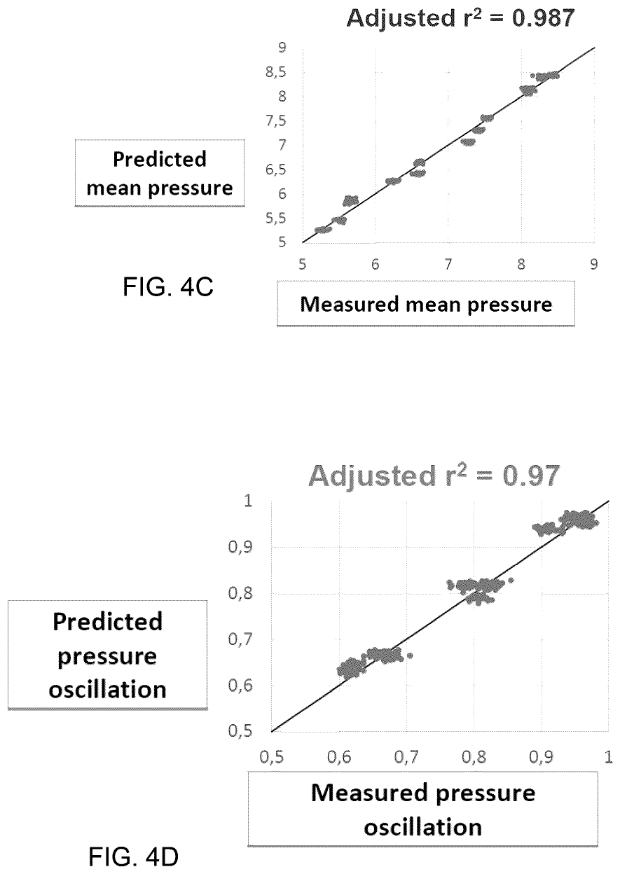

Referring now to FIG. 4A, a graph presents a variation of an amplitude of the sound measured by the sensor 32 as a function of a mean of the pressures delivered to the patient P. The results plotted are for three different flow rates: 6 L/min shown with light dots, 8 L/min shown with darker dots, and 10 L/min shown with dark dots. The results are plotted for four different pressures expressed in centimeter of water. As one can reckon from this graph, as the flow rate increases, an increase in both mean delivered pressures and amplitude of bubble sounds is observed. A correlation factor r of about 0.9 has been computed, which denotes a good correlation between the sound of the bubbles B and the pressure delivered to the patient P.

Referring now to FIG. 4B, a graph presents a variation of an amplitude of the sound measured by the sensor 32 as a function of pressure oscillations provided to the patient P. The results are for a pressure of 8 centimeter of water for three different flow rates. As one can reckon from this graph, a high correlation is found between the amplitude of the sound and the pressure oscillations. A correlation factor r of 0.95 has been computed.

Referring now to FIG. 4C, a graph presents a variation of a predicted mean pressure based on the amplitude of the sound of the bubbles B compared to a measured mean pressure. This graph shows a strong correlation between the two (r²=0.987). Indeed, the addition of the set CPAP level to the sound amplitude metrics improved the prediction of the mean delivered pressures. On this graph, the mean pressure predicted by a model, shown on the y-axis, highly correlated with the measured mean pressure, shown on the x-axis, with an adjusted r-squared of 0.987. The model is based on a multivariate linear regression model.

Referring to FIG. 4D, a graph presents a variation of a predicted pressure oscillation as a function of a measured pressure oscillation. The predicted oscillated pressure predicted by the model highly correlated with the measured pressure oscillation, with an adjusted r-squared of 0.97.

FIGS. 4A to 4D demonstrate the correlation between the sound generated by the bubbles B and the pressure of the air delivered to the patient P. This discovery by the inventors of the present disclosure implies that monitoring the sound generated by the bubbles may effectively be used as a surrogate for measure a pressure of the air supplied to the patient P. As mentioned above, measuring the pressure requires complex, fragile, and expensive sensors that require calibration.

### Method

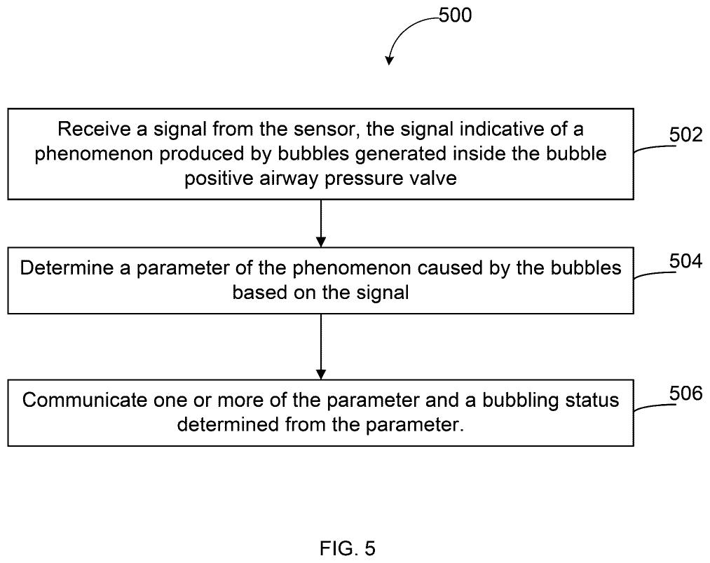

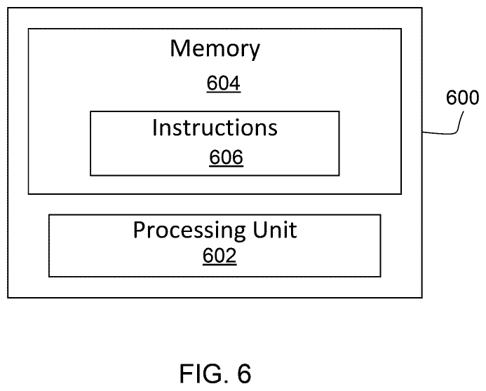

Referring now to FIG. 5, a method for monitoring the CPAP device 10 is shown at 500. The method 500 includes receiving a signal from a sensor 32, the signal indicative of a phenomenon produced by bubbles generated inside the bubble generator 20 at 502; determining a parameter of the phenomenon caused by the bubbles based on the signal at 504; and communicating the parameter and/or a bubbling status determined from the parameter at 506.

In the context of the present disclosure, a “bubbling status” refers to describing the pressure being delivered, the range or magnitude of the pressure oscillations, and the degree of leak in the system, etc. In the present disclosure, the phenomenon generated by the bubbles in the bubble generator 20, such as the vibrations, the noise, and so on, is used to estimate pressure, pressure oscillations, and a degree of leakage in the system. In other words, sound/vibrations or other phenomena are used to estimate the bubbling status. The bubbling sounds/vibrations (or other phenomena) are used to estimate pressure, pressure oscillations, and leaks in the system. All of which are surrogates of effective CPAP delivery and optimal bubbling.

As described above, the phenomenon may include, for instance, noise generated by the bubbles, vibrations generated by the bubbles, fluctuations on a surface S of the liquid in the container 21, fluctuations of a height of the level of the liquid in the container 21, movements on the surface of the liquid, and so on. The parameter may include, for instance, one or more of an amplitude of the sound generated by the bubbles B, a frequency of the sound generated by the bubbles B, an amplitude or pitch of the vibrations generated by the bubbles B on the container 21, a frequency of the vibrations generated by the bubbles B, a measure of the variations in a shape of the surface S of the liquid (e.g., waves when the bubbles B reach the surface S), a measure of the fluctuation in the height of the surface S caused by the bubbles B reaching the surface S, a measure of the movements on the surface S of the liquid, a wavelength of the sound generated by the bubbles B, and so on.

The method 500 may include determining the bubbling status based on the parameter. The bubbling status is indicative of adequate bubbling or inadequate bubbling. In some embodiments, adequate bubbling occurs when the parameter is within a desired range and inadequate bubbling occurs when the parameter is outside the desired range. The desired range may be, for instance, a desired range of the amplitude of the noise.

The communicating of the parameter and/or the bubbling status at 506 may include, for instance, visually displaying the parameter and/or the bubbling status on the display 33A. The communicating may also, or alternately, include issuing an alert (audible or otherwise) when the bubbling status is indicative of inadequate bubbling. The alert may be a visual alert, such as a red light, a flashing indicator, and so on. The alert may be an audible alert, such as a beeping sound, a vibration, and so on. Alternately, the communication of the parameter and/or the bubbling status may include an electronic notification sent to hand-held electronic devices (e.g., phones) of the appropriate personnel (e.g., nurses, doctors, operators of the device, etc.).

In the embodiment shown, the sensor 32 is a sound sensor and the parameter of the bubbles B is an amplitude of a sound generated by the bubbles B. The method 500 includes issuing the alert when the amplitude is below an amplitude threshold.

The method 500 may include receiving a signal from a second sensor, referred to as the ambient sensor 34. The ambient sensor 34 is oriented towards an environment outside the container 21 of the bubble generator 20 in which the bubbles B are generated. The method may include receive a first signal from the sensor 32 and a second signal from the ambient sensor 34. The second signal is indicative of ambient noises generated in the environment. The method 500 may then include correcting the first signal received from the sensor 32 using the second signal to obtain the signal in which the ambient noises are substantially removed from the first signal.

The method 500 may include collecting data about the bubbling for a given time period. For instance, the device may keep in memory the data for the past 24 hours. This data may include one or more of: the bubbling status, the signal, and the parameter determined from the signal.

### Experimental Example

In a particular example, frequency properties of bubbling sounds produced by the bubble CPAP (bCPAP) device 10 were collected and measured in a test system comprising neonatal manikins, and tests were conducted at different settings.

The bCPAP system was connected to a term or preterm manikin. A Millar Mikro-Cath™ pressure catheter was inserted into the inspiratory limb of the device 10 to measure the pressure to ensure near-zero leaks. A Lavalier™ microphone was clipped next to the water canister of the device 10 to capture bubbling sounds. Sound signals were recorded in a quiet NICU room for different combinations of CPAP pressures (5, 6, 7, 8 cmH2O) and flow rates (6,8,10 L/min), for 5 minutes and for a total of 12 experiments. This was repeated twice with each manikin. The first 2 seconds were removed to eliminate transients. Audio recordings were decimated, high pass filtered (100 Hz cutoff) to remove low frequency noise, and the power spectrum computed. Pearson correlation coefficients (r) between summed normalized power and increasing bCPAP pressure/flow were computed for different frequency ranges.

bCPAP bubbling sounds showed frequency components between 250-10,000 Hz, with more than 80% of the power below 2 kHz. When pressure or flow was increased independently, the power in all frequency ranges increased with high correlations (r=0.80-0.99). The frequency range between 750-2000 Hz showed the highest correlations between summed bCPAP power and increasing pressure/flow (r=0.92-0.99, FIG. 3). Notably, power changes were of greater magnitude with increasing pressure than with flow. Results for both preterm and term manikins were similar.

Accordingly, in this experimental example, the frequency content of bCPAP bubbling sounds were consistently between 250-10,000 Hz, with 80% of the power below 2000 Hz. Power was highly correlated with increasing flow/pressure across all frequency ranges, especially between 750-2000 Hz.

### Computing Device

With reference to FIG. 5, an example of a computing device 600 is illustrated. For simplicity only one computing device is shown but the system may include more computing devices operable to exchange data. The computing devices may be the same or different types of devices. The controller 31 may be implemented with one or more computing devices 600.

The computing device 600 comprises a processing unit 602 and a memory 604 which has stored therein computer-executable instructions 606. The processing unit 602 may comprise any suitable devices configured to implement the method 500 such that instructions 606, when executed by the computing device 600 or other programmable apparatus, may cause the functions/acts/steps performed as part of the method 500 as described herein to be executed. The processing unit 602 may comprise, for example, any type of general-purpose microprocessor or microcontroller, a digital signal processing (DSP) processor, a central processing unit (CPU), an integrated circuit, a field programmable gate array (FPGA), a reconfigurable processor, other suitably programmed or programmable logic circuits, or any combination thereof.

The memory 604 may comprise any suitable known or other machine-readable storage medium. The memory 604 may comprise non-transitory computer readable storage medium, for example, but not limited to, an electronic, magnetic, optical, electromagnetic, infrared, or semiconductor system, apparatus, or device, or any suitable combination of the foregoing. The memory 604 may include a suitable combination of any type of computer memory that is located either internally or externally to device, for example random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Memory 604 may comprise any storage means (e.g., devices) suitable for retrievably storing machine-readable instructions 606 executable by processing unit 602.

The methods and systems for monitoring the CPAP device 10 described herein may be implemented in a high-level procedural or object-oriented programming or scripting language, or a combination thereof, to communicate with or assist in the operation of a computer system, for example the computing device 600. Alternatively, the methods and systems for monitoring the CPAP device 10 may be implemented in assembly or machine language. The language may be a compiled or interpreted language. Program code for implementing the methods and systems for monitoring the CPAP device 10 may be stored on a storage media or a device, for example a ROM, a magnetic disk, an optical disc, a flash drive, or any other suitable storage media or device. The program code may be readable by a general or special-purpose programmable computer for configuring and operating the computer when the storage media or device is read by the computer to perform the procedures described herein. Embodiments of the methods and systems for monitoring the CPAP device 10 may also be considered to be implemented by way of a non-transitory computer-readable storage medium having a computer program stored thereon. The computer program may comprise computer-readable instructions which cause a computer, or more specifically the processing unit 602 of the computing device 600, to operate in a specific and predefined manner to perform the functions described herein, for example those described in the method 500.

Computer-executable instructions may be in many forms, including program modules, executed by one or more computers or other devices. Generally, program modules include routines, programs, objects, components, data structures, etc., that perform particular tasks or implement particular abstract data types. Typically, the functionality of the program modules may be combined or distributed as desired in various embodiments.

The embodiments described herein are implemented by physical computer hardware, including computing devices, servers, receivers, transmitters, processors, memory, displays, and networks. The embodiments described herein provide useful physical machines and particularly configured computer hardware arrangements. The embodiments described herein are directed to electronic machines and methods implemented by electronic machines adapted for processing and transforming electromagnetic signals which represent various types of information. The embodiments described herein pervasively and integrally relate to machines, and their uses; and the embodiments described herein have no meaning or practical applicability outside their use with computer hardware, machines, and various hardware components. Substituting the physical hardware particularly configured to implement various acts for non-physical hardware, using mental steps for example, may substantially affect the way the embodiments work. Such computer hardware limitations are clearly essential elements of the embodiments described herein, and they cannot be omitted or substituted for mental means without having a material effect on the operation and structure of the embodiments described herein. The computer hardware is essential to implement the various embodiments described herein and is not merely used to perform steps expeditiously and in an efficient manner.

The term “connected” or “coupled to” may include both direct coupling (in which two elements that are coupled to each other contact each other) and indirect coupling (in which at least one additional element is located between the two elements).

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

It is noted that various connections are set forth between elements in the preceding description and in the drawings. It is noted that these connections are general and, unless specified otherwise, may be direct or indirect and that this specification is not intended to be limiting in this respect. A coupling between two or more entities may refer to a direct connection or an indirect connection. An indirect connection may incorporate one or more intervening entities. The term “connected” or “coupled to” may therefore include both direct coupling (in which two elements that are coupled to each other contact each other) and indirect coupling (in which at least one additional element is located between the two elements).

It is further noted that various method or process steps for embodiments of the present disclosure are described in the following description and drawings. The description may present the method and/or process steps as a particular sequence. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the description should not be construed as a limitation.

Furthermore, no element, component, or method step in the present disclosure is intended to be dedicated to the public regardless of whether the element, component, or method step is explicitly recited in the claims. As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

While various aspects of the present disclosure have been disclosed, it will be apparent to those of ordinary skill in the art that many more embodiments and implementations are possible within the scope of the present disclosure. For example, the present disclosure as described herein includes several aspects and embodiments that include particular features. Although these particular features may be described individually, it is within the scope of the present disclosure that some or all of these features may be combined with any one of the aspects and remain within the scope of the present disclosure. References to “various embodiments,” “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. The use of the indefinite article “a” as used herein with reference to a particular element is intended to encompass “one or more” such elements, and similarly the use of the definite article “the” in reference to a particular element is not intended to exclude the possibility that multiple of such elements may be present.

The embodiments described in this document provide non-limiting examples of possible implementations of the present technology. Upon review of the present disclosure, a person of ordinary skill in the art will recognize that changes may be made to the embodiments described herein without departing from the scope of the present technology. Yet further modifications could be implemented by a person of ordinary skill in the art in view of the present disclosure, which modifications would be within the scope of the present technology.

1. A lung function monitoring system for monitoring lung function of a patient, comprising:
a bubble continuous positive airway pressure (CPAP) device including:
a pressurized gas source of a breathable gas containing oxygen;
an inspiratory conduit fluidly connected to the pressurized gas source and operatively connectable to a patient for supplying air to the patient;
an expiratory conduit fluidly connectable to the patient for receiving exhaled gases that are exhaled by the patient;
a bubble generator including a fluid container connected to the expiratory conduit downstream of the patient, the bubble generator configured for generating bubbles in a liquid within the fluid container using the exhaled gases flowing in the expiratory conduit; and
a monitoring system having:
a sound sensor having a sensing range, the fluid container of the bubble generator located within the sensing range of the sound sensor; and
a controller operatively connected to the sound sensor, the controller having a processing unit and a computer-readable medium having instructions stored thereon and executable by the processing unit to cause the processing unit to:
receive a sound signal from the sound sensor that is indicative of sound generated by bubbles within the liquid of the fluid container;
determine a parameter of the sound generated by the bubbles based on the sound signal; and
determine a property representative of the lung function of the patient using the parameter of the sound, the property including lung compliance and/or airway resistance of the patient.

2. The lung function monitoring system of claim 1, wherein the parameter of the sound generated by the bubbles includes an amplitude and/or a frequency of the sound generated.

3. The lung function monitoring system of claim 1, wherein the parameter of the sound generated by the bubbles includes frequency oscillation of the sound, the frequency oscillation being representative of pressure oscillations produced by the lung function monitoring system and the patient.

4. The lung function monitoring system of claim 1, wherein the computer-readable medium comprises instructions stored thereon executable by the processing unit to: determine one or more dominant frequencies of the bubbles and/or the exhaled gases flowing in the expiratory conduit.

5. The lung function monitoring system of claim 1, wherein the computer-readable medium comprises instructions stored thereon executable by the processing unit to cause the processing unit to: determine a bubbling status based on the parameter, the bubbling status indicative of adequate bubbling when the parameter is within a desired range and indicative of inadequate bubbling when the parameter is outside the desired range.

6. The lung function monitoring system of claim 5, wherein the computer-readable medium comprises instructions stored thereon executable by the processing unit to: issue an alert when the bubbling status is indicative of inadequate bubbling.

7. The lung function monitoring system of claim 1, wherein the sound sensor includes a microphone, an accelerometer configured to capture vibrations, or a micro-electro-mechanical system (MEMS) microphone.

8. The lung function monitoring system of claim 1, wherein the sound sensor is attached directly to the fluid container of the bubble generator.

9. The lung function monitoring system of claim 1, comprising a second sound sensor oriented towards an environment outside the fluid container of the bubble generator, the computer-readable medium further comprising instructions stored thereon executable by the processing unit to cause the processing unit to:
receive the sound signal from the sound sensor and a second sound signal from the second sound sensor, the second sound signal indicative of ambient noises generated in the environment; and
correct the sound signal received from the sound sensor using the second sound signal to obtain a corrected sound signal in which the ambient noises are substantially removed from the sound signal.

10. The lung function monitoring system of claim 1, wherein the computer-readable medium comprises instructions stored thereon executable by the processing unit to cause the processing unit to: communicate and continuously update the property representative of the lung function of the patient in real time.

11. The lung function monitoring system of claim 1, wherein the computer-readable medium comprises instructions stored thereon executable by the processing unit to cause the processing unit to: monitor the lung function of the patient over a period of time, by determining a change in the parameter of the sound generated by the bubbles over the period of time, and therefore a change in the property representative of the lung function over the period of time.

12. A monitoring system for a lung function monitoring system, the lung function monitoring system being operable to monitor lung function of a patient and including a bubble continuous positive airway pressure (CPAP) device, the monitoring system comprising:
a sound sensor having a sensing range, a bubble generator of the bubble CPAP device being adapted to be located within the sensing range of the sensor; and
a controller operatively connected to the sound sensor, the controller having a processing unit and a computer-readable medium having instructions stored thereon and executable by the processing unit to cause the processing unit to:
receive a sound signal from the sound sensor, the sound signal being indicative of sound generated by bubbles within liquid contained in a fluid container of the bubble generator;
determine, based on the sound signal, a parameter of the sound generated by the bubbles;
determine a property representative of lung function of the patient based on the parameter of the sound generated; and
communicate the property representative of the lung function and/or the parameter of the sound generated by the bubbles.

13. The monitoring system of claim 12, wherein the parameter of the sound generated by the bubbles includes an amplitude and/or a frequency of the sound.

14. The monitoring system of claim 12, wherein the parameter of the sound generated by the bubbles includes frequency oscillation of the sound, the frequency oscillation being representative of pressure oscillations produced by the lung function monitoring system and the patient.

15. The monitoring system of claim 12, wherein the computer-readable medium comprises instructions stored thereon executable by the processing unit to: determine one or more dominant frequencies of the bubbles.

16. The monitoring system of claim 12, wherein the sound sensor is a microphone or a micro-electro-mechanical system (MEMS) microphone.

17. The monitoring system of claim 12, comprising a second sound sensor oriented towards an environment outside the fluid container of the bubble generator, the computer-readable medium further comprising instructions stored thereon executable by the processing unit to cause the processing unit to:
receive the sound signal from the sound sensor and a second sound signal from the second sound sensor, the second sound signal indicative of ambient noises generated in the environment; and
correct the sound signal received from the sound sensor using the second sound signal to obtain a corrected signal in which the ambient noises are substantially removed from the sound signal.

18. A method of monitoring lung function of a patient using a bubble continuous positive airway pressure (CPAP) device, the method comprising:
capturing a sound signal representative of sound produced by bubbles within a liquid of a fluid container of the bubble CPAP device, the bubbles being generated in the liquid by exhaled gases from the patient;
processing the sound signal to determine a parameter of the sound produced by the bubbles; and
determining a property representative of the lung function of the patient using the parameter of the sound generated by the bubbles, the property including lung compliance and/or airway resistance of the patient.

19. The method of claim 18, further comprising measuring a frequency oscillation of the sound signal, and determining pressure oscillations present in the exhaled gases of the patient based on the frequency oscillation of the sound signal.

20. The method of claim 18, further comprising determining: one or more dominant frequencies of the sound signal produced by the bubbles; an amplitude of the sound signal produced by the bubbles; and/or a frequency of the sound signal produced by the bubbles.
